(19) United States
(12) Patent Application Publication
(10) Pub. No.: US 2024/0159846 A1
(43) Pub. Date: May 16, 2024

(54) WIRELESS MAGNETIC RESONANCE IMAGING (MRI) COIL WITH COIL FUNCTIONALITY THAT IS NOT RELIANT ON COIL CONTROL SIGNALS FROM THE MRI SCANNER

(71) Applicant: KONINKLIJKE PHILIPS N.V., EINDHOVEN (NL)

(72) Inventors: Scott Bradley King, Gainsville, FL (US); Alton Keel, Gainsville, FL (US); Arne Reykowski, Newberry, FL (US); Timothy Caine Ortiz, Alachua, FL (US); Paul Franz Redder, Newberry, FL (US); Rodrigo Calderon Rico, Newberry, FL (US); Tracy Allyn Wynn, Gainsville, FL (US); Olli Tapio Friman, Eindhoven (NL)

(21) Appl. No.: 18/283,826

(22) PCT Filed: Mar. 18, 2022
(86) PCT No.: PCT/EP2022/057107

§ 371 Date: Sep. 25, 2023

Related U.S. Application Data
(60) Provisional application No. 63/166,305, filed on Mar. 26, 2021.

(51) Int. Cl. G01R 33/36; A61B 5/055; G06T 1/00; G06T 7/00

(52) U.S. Cl. CPC G01R 33/3692; A61B 5/055; G06T 1/0007; G06T 7/00; G06T 2207/10088

(57) ABSTRACT

A magnetic resonance (MR) receive coil (18) includes at least one MR coil element (22) configured to receive MR signals excited in a subject disposed in an MR imaging device (10); an antenna (22, 28) comprising the at least one MR coil element (22) or another antenna (28) that is different from the at least one MR coil element; and electronics (24) configured to detect reception of an electromagnetic pulse of interest by the antenna and to perform a coil function based on the detection. The electromagnetic pulse of interest is a radio frequency (RF) pulse generated by the MR imaging device or a magnetic field gradient pulse generated by the MR imaging device.

### FIELD

The following relates generally to the magnetic resonance (MR) imaging arts, MR coil arts, MR radio frequency (RF) system arts, MR signal acquisition arts, MR clock synchronization arts, and related arts.

### BACKGROUND

Magnetic resonance (MR) imaging entails placing a subject (e.g., medical patient, veterinary subject, archaeological mummy, et cetera) in a static magnetic field (often referred to as a B₀ field) and exciting nuclear magnetic resonance in the subject and then detecting the excited magnetic resonance. For imaging, the excited MR is spatially encoded with respect to location, phase, and/or frequency by superimposing magnetic field gradients on the static B₀ magnetic field during the excitation, during a time interval between MR excitation and MR readout, and/or during the MR readout. In a typical design, the MR imaging device (sometimes referred to as an MRI scanner) includes a housing with a central bore within which the MR examination region is located. The static B₀ magnetic field is produced by solenoidal magnet windings wrapped around the central bore and housed within the MRI scanner housing. These solenoidal magnet windings are often superconducting windings in modern MRI scanners, and the housing includes a liquid helium (LHe) reservoir cooling the superconducting windings. Magnetic field gradient coils are also disposed in the housing around the central bore.

To provide the MR excitation in the case of a human subject, a body coil is commonly used, which is typically a cylindrical birdcage coil, TEM coil, or some variant thereof that is installed concentrically around the bore. Alternatively, a local coil positioned near the body anatomy to be imaged is used for excitation. MR readout is usually performed using a local MR receive coil positioned near the anatomy to be imaged. The local MR receive coil and the local MR excitation coil readout (if used) may be the same coil, or different coils. For various reasons, the MR receive coil (and MR excitation coil, if used) may comprise an MR coil that includes one or more coil elements, with each coil element typically configured as a loop coil, although other coil element designs are known.

In present coil designs, an MRI system sends substantial control information to the radio frequency receive coil, so as to manage the coil operations at a low level. For example, the MRI system may send instructions to detune and send an acknowledgement back to the system, followed by an instruction to tune and send back an acknowledgement, followed by instructions to start and stop acquisition. This increases coil architecture complexity, and the requisite detailed communication is particularly problematic in the case of a wireless coil due to large amounts of radio frequency interference in the MRI room. Additionally, the coil must be designed for the communication protocols of the specific MRI system, so that different coil models may need to be manufactured for MRI systems from different vendors, and possibly even for different models of MRI systems from the same vendor.

The following discloses certain improvements to overcome these problems and others.

### SUMMARY

In some embodiments disclosed herein, a MR receive coil includes at least one MR coil element configured to receive MR signals excited in a subject disposed in an MR imaging device; an antenna comprising the at least one MR coil element or another antenna that is different from the at least one MR coil element; and electronics configured to detect reception of an electromagnetic pulse of interest by the antenna and to perform a coil function based on the detection. The electromagnetic pulse of interest is a RF pulse generated by the MR imaging device or a magnetic field gradient pulse generated by the MR imaging device.

In some embodiments disclosed herein, a MR receive coil includes at least one MR coil element configured to receive MR signals excited in a subject disposed in an MR imaging device; an antenna comprising the at least one MR coil element or another antenna that is different from the at least one MR coil element; and electronics configured to detect reception of an electromagnetic pulse of interest by the antenna and to perform a coil function based on the detection comprising synchronizing acquisition of MR imaging data using the at least one MR coil element at a time determined based on a time of the detection. The electromagnetic pulse of interest is a RF pulse generated by the MR imaging device or a magnetic field gradient pulse generated by the MR imaging device.

In some embodiments disclosed herein, a MR imaging method includes receiving, via a data communication link, MR readout instructions; receiving, via an antenna a RF pulse generated by an MR imaging device; detecting, via electronics, reception of the RF pulse by the antenna; and upon the detecting, performing a coil function in accordance with the MR readout instructions.

One advantage resides in providing an MRI coil with coil functionality time synchronization with the MRI scanner that is not reliant on coil control signals from the MRI scanner.

Another advantage resides in providing such coil functionality time synchronization while retaining the capability of using the MRI coil in a wide range of different MRI pulse sequences.

Another advantage resides in a simplified MRI coil architecture.

Another advantage resides in a simplified communication path between an MRI system and an MRI coil.

Another advantage resides in synchronizing clocks between an MRI system and an MRI coil.

Another advantage resides in providing an MRI coil to be used with an MRI imaging system regardless of a vendor of the MM system.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a magnetic resonance (MR) imaging device including a MR coil element in accordance with the present disclosure.

FIG. 2 shows experimental data for the device of FIG. 1.

FIG. 3 diagrammatically illustrates an MRI imaging method using the device of FIG. 1.

### DETAILED DESCRIPTION

The following discloses an improved MRI receive coil communication approach, which eliminates extraneous communications and makes the receive coil an independent, and optionally vendor-agnostic, actor. To do so, the pulse sequence may be preloaded onto the receive coil. To make this vendor-agnostic, a format conversion program may be employed to convert a pulse sequence file generated by an MRI system of a specific vendor to a vendor-agnostic pulse sequence format, which is then loaded onto the receive coil. As this is being done before the imaging, it can use an asynchronous communication path, e.g., a wired USB cable, wireless Bluetooth connection, or the like. In other examples, the pulse sequence may not be preloaded onto the receive coil. Instead, the pulse sequence can be implemented onto the receive coil via a machine-learning (ML) process incorporated into the transmit pulse detected signals, along with other detected RF receive signals and magnetic field (i.e., gradient) information.

The receive coil with the pulse sequence preloaded is then positioned respective to the target patient anatomy as per usual patient loading, and the MRI sequence is started. (Alternatively, the coil could first be positioned and then the pulse sequence preloaded, e.g., by a wireless Bluetooth connection, prior to starting the imaging).

In some embodiments disclosed herein, the receive coil is further modified to detect an RF pulse (i.e., Tx pulse) and to correlate the detected Tx pulse with the Tx pulse shape expected based on the preloaded pulse sequence. This can be done using any suitable signal correlation method, such as a matched filter, complex correlator, or the like. The detected RF pulse may be the RF pulse that excites initial magnetic resonance (MR), or may be an RF pulse that manipulates the excited MR, or an additional RF pulse dedicated for such correlation-based detection and time alignment, for example delivered by the RF excitation system of the MR imaging system or device. For example, a dedicated RF pulse could optionally be delivered at a frequency different from the system Larmor frequency and not intended to excite or manipulate the MR signal being imaged. For example, in some spin echo sequences a 90° excitation RF pulse excites the MR at a reference time t=0; then, at a time t=TE/2 (where TE is the echo time, also sometimes referred to as the time-to-echo) a rephasing or refocusing 180° RF pulse is applied that rephases or refocuses the MR spins so as to produce a spin echo at a time t=TE. Image acquisition is then performed at the time t=TE after the initial 90° excitation RF pulse (or, equivalently, after at time interval TE/2 from the rephasing 180° RF pulse). This is merely an illustrative example. There are several choices for the antenna to be used to detect the Tx pulse. In one approach, a small pickup coil is added to the receive coil for this purpose. In another embodiment, if the coil elements have automatic gain control (AGC) then one or more coil elements can be used to detect the Tx pulse. In initial experiments, it was found that even if the coil elements have diode-based detuning, depending on the coil design there may nonetheless be enough residual signal on the detuned coil to detect the RF excitation (Tx) pulse shape.

The detection of a Tx pulse that correlates with the expected Tx pulse shape serves as a trigger signal. For example, a coil function such as starting acquisition of MR imaging data (also referred to as readout) using the MR coil element may be performed at a time determined based on a time of the detection of the Tx pulse. In the previous example of a spin echo sequence, if the detection is of reception of the initial 90° excitation RF pulse then the readout suitably begins at the time TE after the 90° excitation RF pulse detection. On the other hand, if the detection is of reception of the refocusing 180° RF pulse then the readout suitably begins at the time TE/2 after the 180° RF pulse detection. At the time determined based on the time of detection of the Tx pulse, the coil executes the preloaded readout sequence, i.e., starts acquisition of MR imaging data. More generally, the detected electromagnetic pulse may be any chosen RF pulse of interest that is generated by the MR imaging device 10 (for example, more particularly by the whole-body RF coil 8) and configured to excite or manipulate the MR signals. In another embodiment, the detected electromagnetic pulse may be a magnetic field gradient pulse generated by the MR imaging device 10 (for example, more particularly by one or more of the magnetic field gradient coils 6) and configured to manipulate the MR signals excited in the subject. The electronics of the MRI coil are configured to detect reception of the electromagnetic pulse of interest and to perform a coil function based on the detection, such as triggering the readout at the time TE, and/or performing operations such as tuning/detuning of the coil elements and sending acknowledgments back to the MRI system as appropriate.

In some embodiments disclosed herein, the preloaded pulse sequence may be parameterized by the same parameters that are adjustable by the MRI technician (e.g., time-to-echo, TE; repetition time, TR; et cetera), and the imaging startup process may include limited communication in which the MRI system sends the parameter values set by the imaging technician to the coil.

In some embodiments disclosed herein, a clock synchronization process is performed. This clock synchronization process relates to synchronizing the oscillator frequency of a local clock of the coil with the oscillator frequency of the MRI system clock, in which the phase of the correlated Tx pulse is used to recover the clock offset.

In other embodiments disclosed herein, a sequence timing is triggered off the detection of a reception of a magnetic field gradient pulse, rather than off an RF pulse. To do this, a magnetic field sensor is added to the local coil to sense the magnetic field. The magnetic field gradient typically has a bandwidth of 20-30 kHz, which translates to typically lower time resolution than is provided by detection of the reception of an RF pulse. Additionally, the idealized (e.g., trapezoidal) gradient pulses often have distorted shapes, e.g., ringing at the transition points.

While described primarily in terms of wireless receive coils, wired receive coils can also benefit from the disclosed approaches, by simplifying the RF coil architecture.

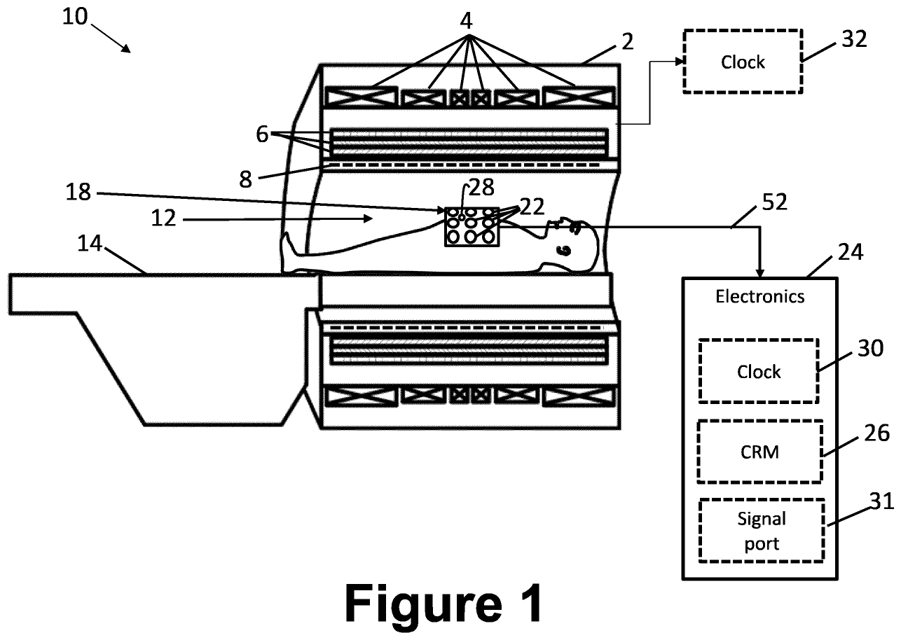

With reference to FIG. 1, an illustrative magnetic resonance (MR) imaging system or device 10 comprises a magnetic resonance (MR) imaging scanner, which in the illustrative example includes a housing or gantry 2 containing various components shown in FIG. 1, such as by way of non-limiting illustrative example a superconducting or resistive magnet 4 generating a static (B₀) magnetic field, magnetic field gradient coils 6 for superimposing magnetic field gradients on the B₀ magnetic field, a whole-body radio frequency (RF) coil 8 for applying RF pulses to excite and/or spatially encode magnetic resonance in an imaging patient disposed in an MR bore 12 or other MR examination region, and/or so forth. The magnet 4 and the gradient coils 6 are arranged concentrically about the bore 12. A robotic patient couch 14 or other patient support enables loading a medical patient, a patient undergoing a medical screening, or other imaging patient into the MR bore 12 for imaging.

The magnetic resonance excited in the imaging subject is read out by an MR receive coil 18 that, in the illustrative embodiment, includes a plurality of MR coil elements 22. (In the limit, the number of coil elements may be 1, that is, the coil may have only a single coil element). Each coil element 22 is a radio frequency antenna for receiving MR signals excited in a subject disposed in the MR imaging device 10. Each coil element 22 typically forms an MR receive channel.

FIG. 1 shows an illustrative MR coil 18 with a single illustrative coil element 22. It will be appreciated that the coil 18 may in general include any number of coil elements 22, e.g., 16 coil elements, 20 coil elements, 32 coil elements, etc. Each coil element 22 is typically part of an MR receive channel that includes the MR coil element 22 configured to receive MR signals in an MR frequency band and a pre-amplifier and often other signal processing electronics. The illustrative coil element 22 is a single loop of copper, copper alloy, or another electrically conductive material, for example formed as a copper layer deposited on a circuit board, plastic sheet, plastic former, or other electrically insulating substrate; or alternatively formed as a freestanding metal loop. However, more generally the coil element 22 may be any suitable antenna capable of coupling with MR signals in the MR frequency band, e.g., a multi-loop coil or otherwise-shaped antenna. In some examples, MR coil 18 including the MR coil element(s) 22 is configured to be disposed in the examination region (i.e., the MR bore 12), as shown in FIG. 1.

The MR coil 18 also includes electronics 24 configured to control operation of the coil element(s) 22. In general, the electronics 24 operate as follows. An antenna receives an electromagnetic pulse of interest generated by the MR imaging device 10, such as an RF pulse. The RF pulse is configured to excite or manipulate the MR signals in the subject. Alternatively, the electromagnetic pulse may be a magnetic field gradient pulse generated by the MR imaging device 10 and configured to manipulate the MR signals excited in the subject. The antenna that receives this RF pulse may, for example, be one of the coil elements 22, or a different coil element 28 of the coil 18 that is different from the coil elements 22 that receive the MR signals. In the case in which the electromagnetic pulse of interest is an RF pulse at the magnetic resonance frequency, the coil elements 22 are typically highly sensitive to that frequency in order to pick up the typically weak MR signals. Hence, the coil elements 22 are typically configured to be detuned during the excitation phase using a detuning circuit (using, for example, a diode, a field effect transistor (FET), micro-electro-mechanism-system (MEMS) sensor, or other suitable active or passively controlled electronic component); or, if automatic gain control (AGC) is provided for the coil elements 22 then the AGC is turned down to a low (or lowest) gain setting during the excitation phase. If a different antenna 28 is used to detect the RF pulse, then it may be designed to detect the RF pulse which is typically much stronger than the MR signals and hence the different antenna 28 can have suitably lower sensitivity. If the electromagnetic pulse of interest is a magnetic field gradient pulse then the different coil element 28 may be designed to pick up that magnetic field gradient pulse, e.g., may be a magnetic field sensor. Alternatively, one or more of the coil elements 22, 28 can be configured to also receive a signal from the magnetic field gradient coils 6. The electronics 24 can also include other components not shown in FIG. 1, such as AGC, pre-amplifiers connected to the coil elements 22 to amplify the (typically weak) received MR signals, an analog-to-digital converter (ADC) to convert the amplified MR signals to digital signals, and/or so forth. These are merely examples.

To detect whether the antenna 22, 28 has received the RF pulse, the electronics 24 include a non-transitory computer readable medium 26 that in one embodiment stores at least one, and optionally a plurality of, predetermined expected electromagnetic pulse shapes (e.g., sinusoidal, trapezoidal, and so forth). The electronics 24 then detect the reception of the RF pulse by the antenna 22, 28 by correlating the RF pulse with the expected electromagnetic pulse shapes. This approach allows for good specificity in detecting a particular electromagnetic pulse, for example being capable of distinguishing between the initial 90° excitation RF pulse or the refocusing 180° RF pulse in the previous example of a spin echo sequence. However, other approaches for detecting whether the antenna 22, 28 has received the RF pulse may be employed. For example, if triggering is off of an initial excitation RF pulse that is the strongest RF pulse in the pulse sequence, then a simple power threshold may be applied to detect this initial excitation RF pulse.

Once the electronics 24 detect that the antenna 22, 28 has received the RF pulse, the electronics perform a coil function of the MR coil 18 based on the detection. For example, the coil function can include synchronizing (for example, starting) acquisition of MR imaging data using the MR coil element 22 at a time determined based on a time of the detection. In another example, the coil function can include performing tuning and/or detuning operations of the MR coil element 22, image acquisition using the MR imaging device 10 (as just mentioned), sending one or more notifications to the MR imaging device, various combinations thereof, and/or so forth.

Preferably, the pulse sequence can be preloaded into the MR receive coil 18. This enables the MR receive coil 18 to be used for a wide variety of different image acquisition sequences. To this end, the electronics 24 can also include a data communication link or signal port 31. For example, in some embodiments, the coil 18 is a wired coil in which case the signal port 31 is suitably an electrical connector or a hardwired connection to an electric cable (e.g., a coaxial cable or a universal serial bus (USB) port and cable). In some embodiments, the coil 18 is a wireless coil and the signal port 31 is a wireless transmitter. In some embodiments, the coil 18 is an optically connected cable and the signal port 31 is an optical transducer/connector assembly where the optical transducer may, for example, be a semiconductor laser diode driven by the processed MR signals and launching light into an optical fiber connected at a connector of the optical transducer/connector assembly. These are merely illustrative examples.

In some embodiments, the MR coil 18 is configured to perform image acquisition based on MR readout instructions. As used herein, the term “MR readout instructions” refer to executable software or firmware program defining an MR readout sequence, and/or one or more parameters of the acquisition of the MR imaging data (e.g., a length of time), and/or so forth. The MR readout instructions can include, for example, a time-to-echo (TE) parameter, a repetition time (TR) parameter for the acquisition of the MR imaging data, and so forth. The electronics 24 are configured to receive the MR readout instructions via the data communication link 31 and store the received MR readout instructions in the non-transitory storage medium 26. Advantageously, this can be done “offline”, that is, before the MR coil 18 is placed onto the patient or other imaging subject, or at least before the imaging sequence actually begins. Then, in accordance with the MR readout instructions, the electronics 24 are configured to perform the acquisition of the MR imaging data in response to the detection of reception of the triggering electromagnetic pulse by the antenna 22, 28.

In the illustrative examples, the electronics 24 are configured to detect reception of an electromagnetic pulse of interest by the antenna and to perform the coil function of synchronizing (for example, starting) acquisition of MR imaging data using the at least one MR coil element 22 at a time determined based on a time of the detection based on the detection. More generally, the electronics 24 may be configured to perform some other coil function based on the detection. For example, the detection may operate by attempting to correlate the electromagnetic pulse of interest received by the antenna 22, 28 with each pulse shape of a set of different predetermined expected electromagnetic pulse shapes. In this case, the detection of the reception will indicate which pulse shape was detected. This information could be used in various ways, such as to identify the vendor and/or model of the MR imaging device 10 or the type of MR excitation coil being used. This identification, in turn, could be used by the MR coil 18 to configure the readout for the particular vendor/model of MR imaging device 10. In another example, the coil function can include applying an autocorrelation or matched filter algorithm to the received signal during the Tx pulse to determine the time offset of highest autocorrelation for pulse sequence time synchronization. This can be performed without prior knowledge of the predetermined electromagnetic pulse that was applied, can be applied. The autocorrelation function Rx (t) is given as Equation 1:

$${R}_{X} ( t ) = {{\int }_{- ∞}}^{∞} x ( τ ) {x}^{*} ( τ - t ) dt \qquad ( 1 )$$

where x(t) is the received signal in time. This measures the similarity of a signal with a time shifted version of itself.

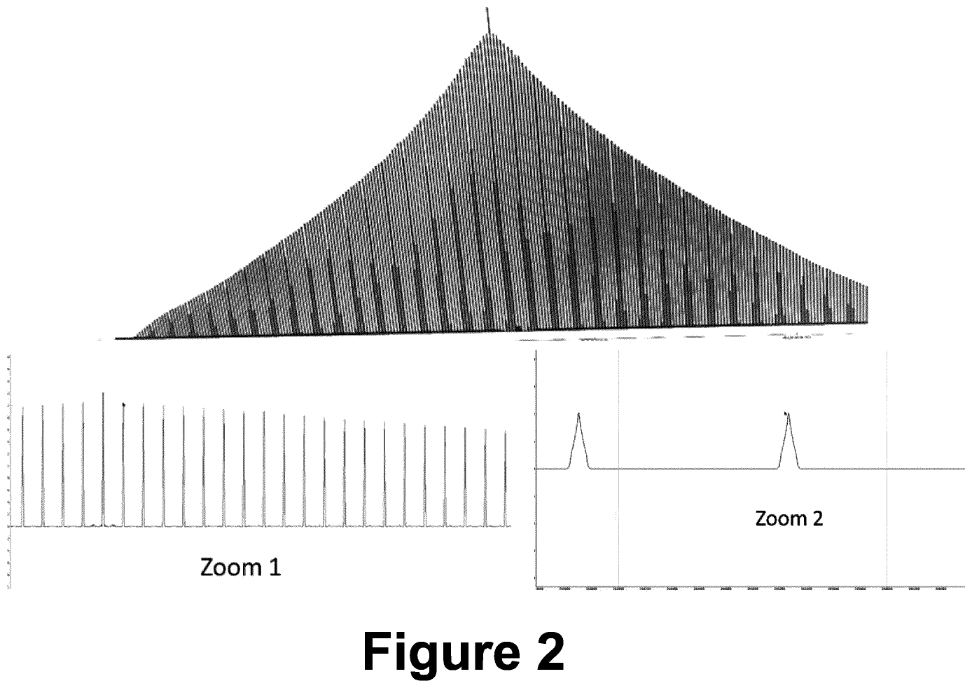

With reference to FIG. 2, experimental data showing autocorrelation of all signals in an entire acquired sequence data set including during Tx, Rx, and delay periods demonstrates that if receiver data is sampled from a receive coil element during the entire pulse sequence, including during transmit, receive, and delay periods, an autocorrelation algorithm can be applied to determine the time of each of the Tx pulses. Alternatively, this correlation can be computed using a set of possible predetermined RF pulse shapes, as a cross-correlation rather than an autocorrelation with itself.

Referring back to FIG. 1, as another example, in some embodiments, the electronics 24 include a local clock 30 and the coil function performed based on the detection comprises synchronizing an oscillator frequency of the local clock with a clock 32 of the MR imaging device 10. In order to determine the clock frequency and phase, non-data aided carrier recovery synchronization techniques used in radio communication, such as Costas loops, can be applied to the detected RF signal during TX phase RF pulse.

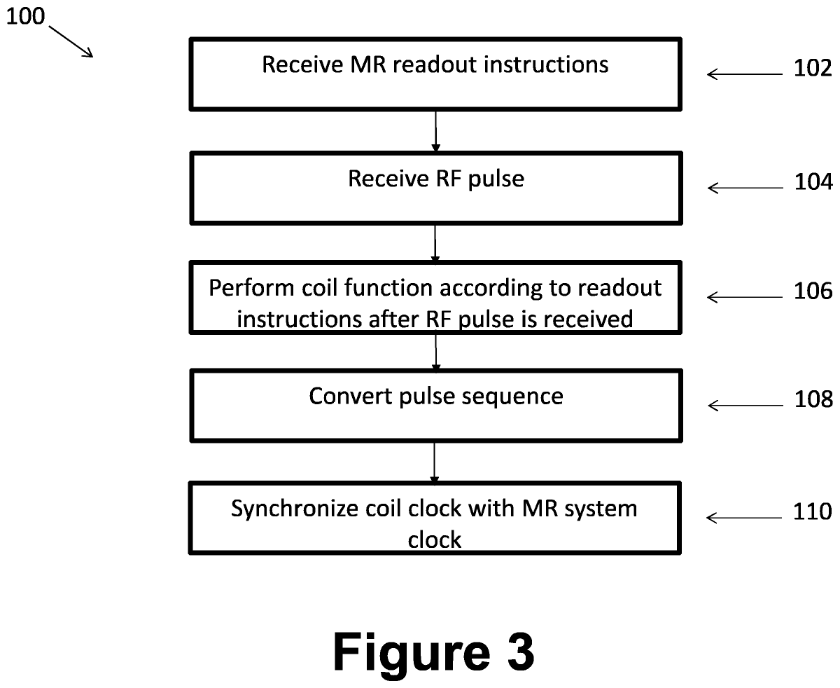

With reference to FIG. 3, and with continuing reference to FIG. 1, an illustrative imaging method 100 using the MR device 10 is diagrammatically shown as a flowchart. To being the method 100, a patient is loaded onto the couch 14 and into the bore 12. The MR coil 18 is placed onto the patient. At an operation 102, the MR readout instructions are received by the data communication link 31. At an operation 104, the RF pulse or other RF pulse of interest is received via the antenna 22, 28, and this reception is detected by the electronics 24. The receiving operations 102 and 104 can be performed concurrently or consecutively (in either order). At an operation 106, upon the receipt of the excitation RF pulse by the antenna 22, 28, the MR coil 18 performs a coil function in accordance with the MR readout instructions.

In an optional operation 108, which performed prior to the MR coil 18 being placed onto the patient, a pulse sequence generated by the MR imaging device can be converted into a pulse sequence having a format independent of the MR imaging device (i.e., a “vendor-agnostic” pulse sequence). This converted pulse sequence is loaded onto the MR coil element 22 of the MR coil 18.

In another optional operation 110, the electronics 24 synchronize the oscillator frequency of the local clock 30 with the clock 32 of the MR imaging device 10.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A magnetic resonance (MR) receive coil, comprising:
at least one MR coil element configured to receive MR signals excited in a subject disposed in an MR imaging device;
an antenna comprising the at least one MR coil element or another antenna that is different from the at least one MR coil element; and
electronics configured to detect reception of an electromagnetic pulse of interest by the antenna and to perform a coil function based on the detection;
wherein the electromagnetic pulse of interest is a radio frequency (RF) pulse generated by the MR imaging device or a magnetic field gradient pulse generated by the MR imaging device.

2. The MR coil of claim 1, wherein the electronics are configured to detect the reception of the electromagnetic pulse of interest by the antenna by correlating the electromagnetic pulse of interest received by the antenna with a predetermined expected electromagnetic pulse shape.

3. The MR coil of claim 1, wherein the electronics are configured to perform the coil function comprising synchronizing acquisition of MR imaging data using the at least one MR coil element at a time determined based on a time of the detection.

4. The MR coil of claim 3, further comprising:
a non-transitory storage medium; and
a data communication link;
wherein the electronics are configured to:
receive MR readout instructions via the data communication link and store the received MR readout instructions in the non-transitory storage medium; and
perform the acquisition of the MR imaging data in accordance with the MR readout instructions stored in the non-transitory storage medium.

5. The MR coil of claim 4, wherein the data communication link is a wireless transceiver or a USB port or an optical fiber connector port.

6. The MR coil of claim 3, wherein the electromagnetic pulse of interest is an RF pulse generated by the MR imaging device and configured to excite the MR signals in the subject.

7. The MR coil of claim 4, wherein the MR readout instructions include:
at least one of a time-to-echo parameter and a repetition time parameter for the acquisition of the MR imaging data.

8. The MR coil of claim 1, wherein the electromagnetic pulse of interest is an RF pulse generated by the MR imaging device and configured to excite the MR signals in the subject, and the electronics are configured to:
perform a least one of tuning operations of the MR coil element, image acquisition, and sending a notification to the MR imaging device.

9. The MR system of claim 1, wherein the antenna comprises the at least one MR coil element.

10. The MR coil of claim 1, wherein the electronics include a local clock and the coil function performed based on the detection comprises synchronizing an oscillator frequency of the local clock with a clock of the MR imaging device.

11. An MR system comprising:
an MR imaging device including a magnet configured to generate a static magnetic field in an examination region and magnetic field gradient coils configured to superimpose magnetic field gradients on the static magnetic field in the examination region; and
an MR coil as set forth in claim 1.

12. A magnetic resonance (MR) receive coil, comprising:
at least one MR coil element configured to receive MR signals excited in a subject disposed in an MR imaging device;
an antenna comprising the at least one MR coil element or another antenna that is different from the at least one MR coil element; and
electronics configured to detect reception of an electromagnetic pulse of interest by the antenna and to perform a coil function based on the detection comprising synchronizing acquisition of MR imaging data using the at least one MR coil element at a time determined based on a time of the detection;
wherein the electromagnetic pulse of interest is a radio frequency (RF) pulse generated by the MR imaging device or a magnetic field gradient pulse generated by the MR imaging device.

13. The MR coil of claim 12, wherein the electronics are configured to detect the reception of the electromagnetic pulse of interest by the antenna by correlating the electromagnetic pulse of interest received by the antenna with a predetermined expected electromagnetic pulse shape.

14. The MR coil of claim 12, further comprising:
a non-transitory storage medium; and
a data communication link;
wherein the electronics are configured to:
receive MR readout instructions via the data communication link and store the received MR readout instructions in the non-transitory storage medium; and
perform the acquisition of the MR imaging data in accordance with the MR readout instructions stored in the non-transitory storage medium.

15. The MR coil of claim 12, wherein the electromagnetic pulse of interest is an RF pulse generated by the MR imaging device and configured to excite the MR signals in the subject.

16. The MR coil of claim 12, wherein the electromagnetic pulse of interest is an RF pulse generated by the MR imaging device and configured to excite the MR signals in the subject, and the electronics are configured to:
perform a least one of tuning operations of the MR coil element, image acquisition, and sending a notification to the MR imaging device.

17. The MR coil of claim 12, wherein the electronics include a local clock and the coil function performed based on the detection comprises synchronizing an oscillator frequency of the local clock with a clock of the MR imaging device.

18. A magnetic resonance (MR) imaging method, comprising:
receiving, via a data communication link, MR readout instructions;
receiving, via an antenna a radiofrequency (RF) pulse generated by an MR imaging device;
detecting, via electronics, reception of the RF pulse by the antenna; and
upon the detecting, performing a coil function in accordance with the MR readout instructions.

19. The MR method of claim 18, further including:
converting a pulse sequence generated by the MR imaging device into a converted pulse sequence format independent of the MR imaging device; and
loading the converted pulse sequence onto a MR coil element.

20. The MR method of claim 18, further including:
synchronizing an oscillator frequency of a local clock of the electronics with a clock of the MR imaging device.
